(19) United States
(12) Patent Application Publication
(10) Pub. No.: US 2017/0319641 A1
(43) Pub. Date: Nov. 9, 2017

(54) MUCOADHESIVE EMOLLIENT

(71) Applicant: BIOGLAN AB, MALMÖ (SE)

(72) Inventors: Birgitta SVENSSON, BUNKEFLOSTRAND (SE); Johan ENGBLOM, LUND (SE)

(21) Appl. No.: 15/525,238

(22) PCT Filed: Nov. 12, 2015
(86) PCT No.: PCT/SE2015/051207

§ 371 Date: May 8, 2017

(30) Foreign Application Priority Data
Nov. 14, 2014 (SE) 1451365-9

(51) Int. Cl. A61K 36/185; A61K 31/25; A61K 9/00; A61K 9/107; A61K 47/26; A61K 47/10

(52) U.S. Cl. CPC A61K 36/185; A61K 47/26; A61K 47/10; A61K 9/0043; A61K 9/1075; A61K 31/25

(57) ABSTRACT

The present invention relates to a microemulsion comprising: 1-98 wt % of at least one polar lipid selected from polyglycerol fatty acid esters; 1-98 wt % of at least one emollient, not being a polyglycerolfatty acid ester; 0.1-3 wt % of at least one polar solvent; and 0-10 wt % of a surfactant, not being a polyglycerol fatty acid ester. The present invention further relates to said microemulsion for use as a medicament, and for use in the treatment and prevention of dry mucosa or prevention of airborne particles reaching topical mucosal membranes of a mammal. The present invention also relates to a liquid composition comprising said microemulsion, and an applicator device comprising said microemulsion.

### TECHNICAL FIELD OF THE INVENTION

The present invention relates to novel lipid based formulations. More particularly the invention relates to moisturizing mucoadhesive formulations for topical use, which are useful for the prevention of symptoms in mammals, where such symptoms are directly or indirectly related to dry mucosa or by airborne particles. The formulations may be formulated as low viscous sprayable microemulsions. The present invention relates to microemulsions, compositions comprising said microemulsion, their use and application devices.

### BACKGROUND OF THE INVENTION

The mucosa constitutes a protective barrier between the body interior and the ambient environment. The tissue consists of globlet, cilia and microvilli cells and other types of cells, blood vessels, nerves and glands. The mucosa is covered by a mucus film released by the glands. Mucus is a viscous fluid that moistens, lubricates, and protects the passages of the digestive and respiratory tracts in the body. Mucus is composed of water (about 95%), epithelial (surface) cells, dead leukocytes, mucin (mucopolysaccharide), and inorganic salts. Mucus in the nose helps to trap dust and other particles, such as pollen, and microorganisms, such as bacteria and viruses, from reaching the lungs. It is continuously transported from the anterior to the posterior part of the nasal cavity with help from the cilia hairs. This mechanism is called mucociliary clearance. The mucus layer is renewed every 15 to 20 minutes under normal conditions.

Air flow through the nose can dehydrate the tissue and result in a dry and irritated mucosa. It may lead to nasal congestion, crusts formation and nose bleed. Dry nose phenomena leading to nosebleed occur more often during the winter, when cold viruses are common and indoor air tends to be drier. The mucosa may require use of moisturizing products (i.e. “moisturizers”) to become comfortable.

Dust particles from polluted air, pollen or microorganisms may cause inflammation in the nose. Rhinitis is an inflammation of the mucous membranes lining in the interior of the nose. It produces symptoms like a stuffy, runny nose, and sneezing. It can be caused by allergies (i.e., allergic rhinitis) or by a cold, injury, or pollutants (i.e., non-allergic rhinitis). Rhinitis is also related to increased permeability of the mucosa.

Pollen, and/or dust mite allergy, allergic rhinitis, is very common. The reaction is initiated by allergens that come in contact with the mucosa. Glucocorticosteroids and antihistamines are commonly used to treat such inflammation. Side effects after using corticosteroids are irritated and crusty nose, and nose bleed. Patients suffering from pollen and mite allergy benefit both from treatments that prevent inflammation and from treatments that moisturise the nose when medication is required.

Topical formulations that enforce the barrier of the nasal mucosa have emerged as preventive measures in the treatment of rhinitis, whereas moisturizers are used for dry nose symptoms. Several types of moisturising and/or barrier enforcing measures have been tested previously, from cellulose film forming agents to gels, creams, ointments and microemulsions. The common mechanism may be that the substrates are applied onto the mucosal surface to provide an extra barrier that prevents the allergen or particles from reaching the mucosa and to hydrate the mucosa. The mechanism behind moisturising the mucosa can be occlusive (compare ointments) or hydration via the vehicle (compare saline solutions).

It is important that the formulation covers a large surface area of the nasal mucosa to be optimal as barrier improving treatment. This can be achieved by compositions that are readily spreading over the surface, which is the case for surface active preparations such as microemulsions and emulsions. Oil solutions, ointments, hydrogels and hydrophilic solutions will not have the same driving force to cover the surface. Larger areas may remain unprotected.

Surface active preparations can entrap pollen and microorganisms due to their surface active properties, and thereby prevent the particles from reaching the mucosa and inhibit the release of allergens or start an infection. The entrapped particles will follow the mucus and swallowed or sneezed out (i.e. are eliminated from the body therewith).

Nasal protective barrier products require a lengthened period of time in the nose to have a practical benefit and not be eliminated immediately by follow the mucus. One advantage with a microemulsion compared to an emulsion is that the microemulsion may have inherent adhesive properties due to thermodynamic driven water sorption from the underlying mucosa. The formulation is pulling water from the nasal mucosa and thereby there is an attractive force that helps the formulation to stay in the nose. This is referred to as mucoadhesion (or bioadhesion), i.e. increased interactions between the mucosa and the product.

Both emulsions and microemulsions may provide emollient effects that prevent desiccation (drying out) of underlying tissue in situ. One advantage of a microemulsion compared to an emulsion is that the former is a thermodynamically stable one phase system. Another advantage of a microemulsion compared to an emulsion is that there is higher probability of forming an intact film by a one-phase system compared to a two-phase system. A bicontinuous or a reversed type of microemulsion has furthermore higher probability of forming an intact film as compared to a direct type (normal oil-in-water type) of microemulsion. Another advantage of a microemulsion compared to an emulsion is that microemulsions are easy to administer to the nose. They are low viscous, sprayable and fast spreading.

Microemulsions that are strongly mucoadhesive through water sorption by the formulation may provide prolonged duration in situ. However, water sorption by the formulation will also desiccate underlying tissue. Strong formulation water sorption may furthermore induce a stinging unpleasant sensation, which tentatively may be attributed to the abovementioned tissue dehydration. Strong initial formulation water sorption and resulting dehydration of the mucosa result in an initially unbalanced water transport leading to an unpleasant stinging sensation upon application even if the formulations are occlusive.

Microemulsions based on glycerol monooleate have been studied extensively (e.g. EP1648412). EP1648412 B1 discloses a microemulsion comprising 20 to 50 wt % monoacyl glyceride, 10-55 wt % polar solvent, 5 to 35% non-polar animal or vegetable oil and at least one surfactant. Such water-in-oil discrete particular microemulsions have been reported to reduce allergen challenge-induced symptoms in patients with seasonal allergic rhinitis as well as nasal symptoms of perennial allergic rhinitis at natural allergen exposure. The microemulsions in EP1648412 are sensitive to temperature fluctuations and phase separates when stored at lower temperature (the water holding capacity is decreasing and some compounds are crystallising below room temperature). The microemulsions in EP1648412 are initially absorbing water so fast that they can be experienced as stinging upon application.

US2012/0100234 A1 [Kulesza & Kats, 2012] disclose methods of promoting improved nasal moisture and reduction in nasal congestion.

The publication by Shrestha L K, Dulle M, Glatter O, Aramaki K. “Structure of polyglycerol oleic acid ester nonionic surfactant reverse micelles in decane: growth control by headgroup size.” Langmuir. 2010 May 18; 26(10):7015-24 show that an added water phase is not a requirement for forming microemulsions when using glycerol esters of technical grade in an oil mixture. Water-in-oil discrete particular microemulsions are formed by using technical grade glycerol ester and oil mixtures only. The larger the headgroup, i.e., the longer glycerol chain, the more the micelles grow and interact. This is related to the spontaneous curvature of the reverse micelles. Increased headgroup size of the amphiphile keeping the lipophilic tail identical decrease the critical packaging parameter which means aggregates with less negative curvature, i.e. larger or elongated particles are formed.

Accordingly, there is a need for new and more efficacious treatments also in this topical area. There is a need for a way of preventing or reducing the abovementioned symptoms associated with rihinitis and dry nose. It would be most desirable if a single composition was effective against a range of such conditions.

There is a need for providing a preparation capable of hydrating mucosa during an extended time, whereby to prevent the above-mentioned problems.

Also, there is a need for providing a preparations that are able to easily spread to form an occlusive film on the mucosa, whereby to prevent the above-mentioned problems.

Furthermore, there is a need for providing preparations that follow the mucus when self-cleaned, swallowed or sneezed out (i.e. are eliminated from the body therewith).

### SUMMARY OF THE INVENTION

The above mentioned problems are solved by the present invention. The formulations of the present invention are designed in such a way that they combine mucoadhesive properties with the ability to moisturize the mucosa on application and over time, and also prevent the undesired initial stinging effect found in conventional products as well as the sensitivity to temperature fluctuations found in conventional products. The formulations may furthermore provide increased hydration to the mucosa even after the formulation has been eliminated from the application site.

These objectives are accomplished by provision of a microemulsion comprising polar lipid, emollient, and at least one polar solvent.

We have surprisingly found that incorporation of a humectant in said microemulsion may promote mucoadhesion due to water sorption from the underlying tissue, and at the same time increase water content in said tissue. Incorporation of humectants may also surprisingly reduce, or eliminate, the stinging sensation frequently observed with mucoadhesive formulations.

The present invention relates in one aspect to providing a microemulsion comprising: 1-98 wt % of at least one polar lipid selected from polyglycerol fatty acid esters; 1-98 wt % of at least one emollient, not being a polyglycerol fatty acid ester; 0.1-35 wt % of at least one polar solvent; 0-10 wt % of a surfactant, not being a polyglycerol fatty acid ester.

In one embodiment the at least one polar lipid is selected from fatty acid esters of diglycerol to hexaglycerol, preferably fatty acid esters of diglycerol to pentaglycerol, preferably fatty acid esters of diglycerol to tetraglycerol.

In one embodiment the fatty acid esters of the polyglycerol fatty acid esters are chosen from oleate, linoleate, and linenololeat, preferably chosen from monooleate, monolinoleate, and monolinenololeat, preferably monooleate.

In one embodiment the polyglycerol fatty acid esters are present in an amount of 20-50 wt %, preferably 25-40 wt %, eg 30-35 wt %, eg 31, 32, 33, 34, 35, 36, 37, 38, 39 or 40 wt %.

In one embodiment the at least one polar solvent is chosen from hydrophilic solvents, preferably water or water solution; glycerol; low molecular weight glycerol derivatives; propylene glycol; low molecular weight derivatives of propylene glycol; low molecular weight alcohol, preferably ethanol, isopropyl alcohol and transcutol; butylene glycol; pentylene glycol; hexylene glycol; dipropylene glycol; propanediol; panthenol; propylene carbonate; and polyethylene glycol.

In one embodiment the at least one polar solvent is present in an amount of 0.5-30 wt %, preferably 1-30 wt %, preferably 1-25 wt %, preferably 5-25 wt %, preferably 5-20 wt %, eg, 10-15, eg 10, 11, 12, 13, 14, 15, 16, 17, 18, 19, 20 wt %.

In one embodiment the at least one emollient is selected from fat, animal or vegetable oils, vegetable butters, wax, silicones, petrolatum and mineral oil; preferably mineral oil, silicone oils, animal or vegetable oils; preferably vegetable oils.

In one embodiment the at least one emollient is selected from the group consisting of low molecular weight linear polysiloxanes and cyclic dimethyl polysiloxanes; liquid paraffin; lanolin oil; vegetable triglyceride containing oil selected from almond, avocado, canola, castor, coconut, corn, evening primrose, jojoba, linseed, olive, safflower, sunflower, sesame, soybean, and wheat germ oil; and extractions of triglycerides with 8 to 10 carbons in the chain.

In one embodiment the at least one emollient is present in an amount of 10-80 wt %, preferably 20-73 wt %, preferably 30-70 wt %, preferably 33-70 wt %.

In one embodiment the at least one surfactant is selected from nonionic surfactants, preferably hydrophilic surfactants, preferably fatty acid ethoxylates esters and ethers, sorbitane monoesters, poloxamers and alkyl glucosides, preferably polysorbate 20, 40, 60 or 80, preferably polysorbate 60 or 80, preferably polysorbate 80.

In one embodiment the at least one surfactant is present in an amount of 0-7 wt %, eg 1, 2, 3, 4, 5, 6, or 7 wt %.

In one embodiment the microemulsion may further comprise a compound chosen from a humectant, not being a polar solvent; an antioxidant, preferably tocopherol; a complexing agent; and a preservative.

In one embodiment the humectant is selected from the group urea, aloe vera, pidolic acid, alfa-hydroxy acids, sorbitol, xylitol, manitol and natural polymers having humectant properties, preferably the natural polymer is hyaluronic acid.

In one embodiment the at least one humectant is present in an amount of 0-20 wt %, preferably 0-10 wt %, preferably 0-5 wt %, eg 1, 2, 3, 4, or 5 wt %.

In one embodiment the microemulsion is an oil-continuous isotropic solution with no aggregates; or has discrete reverse micelles with a hydrophilic core and a hydrophobic continuous phase.

The present invention relates in one aspect to providing said microemulsion for use as a medicament.

The present invention relates in one aspect to providing said microemulsion for use in the treatment and prevention of dry mucosa or prevention of airborne particles reaching topical mucosal membranes of a mammal.

The present invention relates in one aspect to providing a microemulsion for use in the treatment and prevention of dry mucosa or prevention of airborne particles reaching topical mucosal membranes of a mammal, wherein the microemulsion is administered in the form of a nasal, buccal, rectal or vaginal application, preferably nasal spray, buccal spray, rectal spray or vaginal spray.

The present invention relates in one aspect to providing a liquid composition comprising said microemulsion.

In one embodiment said composition is in a form suitable for nasal, buccal, rectal or vaginal administration.

In one embodiment said composition is sprayable.

The present invention relates in one aspect to providing an applicator device comprising said microemulsion.

In one embodiment said applicator device is a nasal applicator device, a buccal applicator device, a rectal applicator device or a vaginal applicator device; preferably a nasal spray device, a buccal spray device, a rectal spray device or a vaginal spray device.

The present invention relates in one aspect to a method of moisturizing nasal, buccal, vaginal and rectal passages, alleviating nasal, buccal, vaginal and rectal dryness and optionally preventing airborne particles reaching (topical) mucosal membranes of a mammal, the method comprising the steps of: providing a nasal, buccal, vaginal and rectal spray solution comprising a microemulsion according to the present invention; and delivering at least one application of the solution into at least one nasal, buccal, vaginal and rectal cavity, e.g. a nostril.

According to one embodiment the at least one application is sprayed into the at least one nostril.

The present invention relates in another aspect to a method of administering a composition comprising a microemulsion according to the present invention to a mammal via the nasal, buccal, vaginal and rectal route.

The microemulsion according to the present invention may be used in the manufacture of a composition for treatment and prevention of a condition related directly or indirectly to dry mucosa or airborne particles.

The microemulsion according to the present invention may be used in the manufacture of a composition for treating and preventing dry mucosa or preventing airborne particles reaching (topical) mucosal membranes of a mammal, by administering said composition to said mucosal membranes.

The microemulsion according to the present invention may be used for application to the mucosa of a mammal, preferably to the nasal, buccal, vaginal and rectal mucosa.

### BRIEF DESCRIPTION OF THE DRAWINGS

FIG. 1 depicts gravimetric evaporation from a thin film of product on a gelatin hydrogel at about 23° C. and 20-40% RH in the surrounding. Reference 1 is sesame oil and Reference 2 is a non-covered hydrogel. The overlapping graph is just an enlarged view.

FIG. 2 depicts gravimetric water uptake in the products at about 23° C. and 97% RH. S1 to S8 are the samples with the compositions according to Example 6.

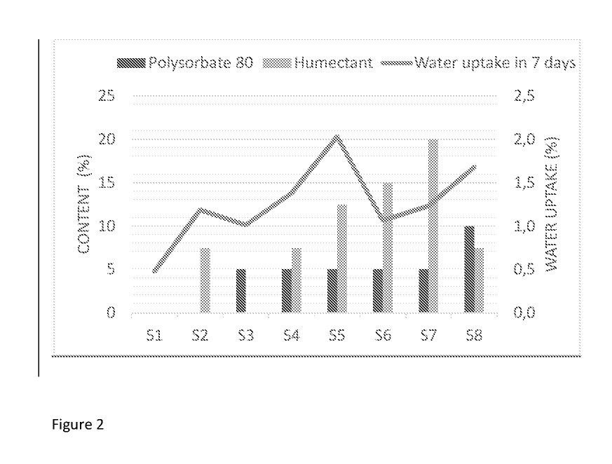

### DETAILED DESCRIPTION OF THE INVENTION

An object of the present invention is to obtain microemulsions that are mucoadhesive and moisturising, sprayable, and with good spreading capacity on mucosal surfaces with an increased stability at lower temperature (no crystallisation or phase separation).

Moisturizers are products used to hydrate tissue, comprising emollients (e.g. petrolatum, mineral oil, fatty acid esters, silicones) and/or humectants (e.g. glycerol, sorbitol, urea, propylene glycol). Both emollient and humectant based formulations require good spreading to form a continuous film in situ, and prolonged contact between the film and the tissue for desired effect (occluding film or uptake in tissue of hydrophilic components which will retain water).

Products administered to the nose are normally easily eliminated by the mucociliary clearance system within 15-20 minutes from application. Furthermore, in case of emollients, petroleum or silicone based oils, sprayed in the nose suffers from a risk of being deposited in the lungs. Accumulation of oil in the lungs may lead to potentially serious inflammation known as lipoid pneumonia. Using a mucoadhesive surface active formulation with body own type of degradable fatty acid based excipients is one way to overcome this risk.

The microemulsions of the present invention are highly useful in themselves, when formulated as compositions suitable for application to a mammalian subject. In another aspect, the invention thus provides a composition suitable for administration to peripheral membrane linings of, for example, the nose, the eyes, the ears, the pharynx, and/or the larynx of a mammal, characterized in that it comprises a pharmaceutically effective amount of a microemulsion, especially a microemulsion of the invention.

In yet still further aspect, the invention provides a microemulsion of the invention for use in therapy and in a still further aspect the invention provides the use of a microemulsion or composition of the invention in the manufacture of a medicament for the treatment or prevention of a disease related directly or indirectly to dry nose or airborne particles.

Herein, the wording “moisture” is defined as the amount of water, i.e., in mol %, volume % or weight %.

The wording “humidity” is herein defined as water chemical potential (μ_{w}), water thermodynamic activity (a_{w}) or relative humidity (% RH=100*a_{w}) where applicable.

The wording “moisturizers” are herein defined as products used to hydrate tissue, comprising emollients (e.g. petrolatum, mineral oil, fatty acid esters, silicones) and/or humectants (e.g. glycerol, sorbitol, urea, propylene glycol, polyethylene glycol).

The wording “mucoadhesive” relates to the ability to adhere to the mucosa. Mucoadhesion can be achieved through water sorption from tissue to the applied formulation. A hygroscopic formulation then absorbs water molecules (i.e. “mass of water, moisture”) from the tissue, driven by the gradient in water thermodynamic activity, a_{w}. Hence, a prerequisite for adhesion is then existence of an a_{w} gradient. The overall gradient in a_{w} (from inside to outside) does not change, but the distribution of the gradient profile may be shifted. Adhesive joint failure will inevitably occur as a result of overhydration of a dosage form.

The wording “micoremulsion” can be defined as a thermodynamically stable dispersion of two immiscible substances, a polar component and an apolar component, e.g. water and oil, which become miscible by a third component, an amphiphilic molecule. The microemulsion is a clear and isotropic solution as the dispersed structure is less than about 100 nm in diameter. There are three basic types of microemulsions; direct (oil dispersed in water, O/W), reversed (water dispersed in oil, W/O) and bicontinuous.

The microemulsions according to the present invention are preferably of the reverse micellar type, W/O, i.e. discrete reverse micelles with a hydrophilic core and a hydrophobic continuous phase. The system could also be an oil-continuous isotropic solution with no discrete aggregates.

The microemulsion according to the present invention contains at least one polar component as one of the immiscible components. Polar components will be present in microemulsions based on glycerol esters as impurities but larger amounts may be wanted for other reasons.

The wording “polar solvent” is a hydrophilic solvent such as water or a water solution, glycerol and low molecular weight glycerol derivatives, propylene glycol and low molecular weight derivatives of propylene glycol, low molecular weight alcohol. One polar solvent can be used alone or as combination between several solvents. The polar solvent(s) is preferably liquid at room temperature, has low toxicity, good stability, high flash point, low vapour pressure and broad solvency in the system for optimal use in the application of the present invention.

An amount of water or water solution soluble in the system will depend on the combination of the other components but preferably the amount is less than about 10% to have good long-term stability of the microemulsion in a wide temperature range.

The amount of other polar solvent than water or water solutions that is soluble in the system will depend on the combination of the other components but preferably the amount is less than about 25% to have good stability of the microemulsion.

Low molecular weight polar solvents such as ethanol, isopropyl alcohol and transcutol increase the solubility of the glycerol esters in the oil and less ordered structure is formed. Low molecular weight solvents will also give clear solutions with good surface spreading characteristics.

Some polar solvents are categorized as having humectants properties, for example glycerol, propylene glycol, butylene glycol, pentylene glycol, hexylene glycol, dipropylene glycol, caprylyl glycol, propanediol, panthenol, and propylene carbonate. Glycerol, propylene glycol, butylene glycol, pentylene glycol, hexylene glycol and caprylyl glycol are a preferred type of solvent due to its humectant properties and it good solubility in the system. These humectants are however herein disclosed as polar solvents in order to better distinguish the formula of the present invention.

The wording “humectant” relates to a substance that promotes retention of moisture. A humectant substance is any one of a group of hygroscopic (water attracting) hydrophilic substances that keep body surfaces moist. Humectants are characterized by containing hydroxyl groups, amines or carboxyl groups and are sometimes esterified.

Herein the present invention may additionally comprise a humectant, which is not a polar solvent. Such humectants may be pidolic acid, alfa-hydroxy acids, sorbitol, xylitol, manitol, and urea. Natural polymers such as hyaluronic acid may also be categorized as such humectants. Hyaluronic acid is a linear polymer of dimeric N-acetyl glucosamine and glucuronic acid. It is present in the skin as part of the extracellular matrix and as a component of collagen and is extremely hydrophilic and biochemically retains water on the skin. Further, humectant substances such as urea or aloe vera containing high concentration of humectant substances may also be included as humectants, which are not polar solvents.

Humectants do not affect the a_{w} gradient, but instead the mass of water at a given a_{w}. Low molecular weight humectants increase mass of water in tissue, but does not affect “humidity (a_{w})”. High molecular weight humectants do not enter tissue; but may contribute with a softening and smoothing feeling.

The microemulsion of the present invention contains an apolar component as one of the immiscible components. This apolar component may be an emollient.

The present invention comprises at least one emollient. The wording “emollient” relates to something that softens or smoothes. Emollients are materials that smooth the surface and make the surface look uniform to the eye and silkier to touch. Emollients are lipophilic and may produce an occlusive film which may promote a steeper a_{w} gradient in tissue i.e. the emollient becomes a barrier to the outside which hampers evaporation from the superficial layer in tissue.

Examples of emollients are: fat and vegetable oils—subgroup of lipids including synthetic or natural glycerides (mono, di or triglyceride), wax—subgroup of lipids including natural or synthetic esters of fatty acids with long chain hydrocarbons, silicones, petrolatum (hard or soft paraffin) and mineral oil (liquid paraffin)—subgroups of lipids including non-vegetable long or short chain hydrocarbons (originating from petroleum).

It is to be noted that the polar lipid selected from polyglycerol fatty acid esters have emollient properties. However, these polar lipids are displayed by themselves as a separate category in the formulations herein.

The emollients are preferably selected from animal or vegetable oils or mineral oils to avoid low temperature crystallization of the microemulsion. Non-vegetable silicone oil such as low molecular weight linear polysiloxanes and cyclic dimethyl polysiloxanes may also be used as well as non-vegetable mineral oil (liquid paraffin), long or short chain hydrocarbons (originating from petroleum).

Emollients of animal or vegetable origin are a preferred embodiment according to the present invention since they are similar to the body own mono/di/triglycerides and phospholipids. Accumulation of oil in the lungs may lead to potentially serious inflammation known as lipoid pneumonia. Using body own type of degradable fatty acid based excipients is one way to overcome this risk.

Animal oil is preferably selected from lanolin oil.

Another preferred emollient is vegetable triglyceride oil with about 18 carbons in the chain and high degree of unsaturation, refined and deodorised. These oils are preferred since they are low irritating and liquid at low temperatures.

Among the vegetable oils, the most preferred oils are the ones that are purified in such a way as they can be regarded as low allergenic.

The vegetable oil preferably selected from almond, avocado, canola (rapeseed), castor (ricinic), coconut, corn (maize), evening primrose, jojoba, linseed, olive, safflower, sunflower, sesame, soybean, and wheat germ oil. Extractions of triglycerides with 8 to 10 carbons in the chain (medium chain triglydcerides, MCT) are also preferred since they have low viscosity and therefore suitable in a sprayable product.

The emollient according to the invention may together with the polar lipid prevent dehydration by forming a protective occlusive barrier.

Amphiphiles are molecules that consist of segments with different preference regarding the solvent. One part of the amphiphile is hydrophilic (polar) and prefers contact with water and other hydrophilic compounds. The other part is hydrophobic (apolar) and prefers contact with oil and other hydrophobic compounds. Amphiphilic molecules are surface active, i.e. are spreading easily on interfaces, e.g. the interface between a body tissue such as mucosa and air. Amphiliphilic molecules can spontaneously self-organize into microscopic structures in presence of polar and/or apolar liquid(s) due to the molecules amphilicity. Isotopic solutions are disordered over long and short distances but can form aggregates in one or two dimensions, so called micellar solutions. Liquid crystalline structures are ordered on long distances but disordered on short. The aggregates can be ordered in two dimensions (hexagonal and lamellar) or three dimensions (cubic).

In the present formulations the amphiphilies disclosed are divided into two categories. The polar lipids being polyglycerol fatty acid esters are one type of amphiphilies used in present formulations but also other types of surfactants may be used in the formulations. Thus, these are dealt with separately in the formulations.

Surfactants are amphiphilic molecules. Herein, the surfactants are excluding polyglycerol fatty acid esters, as they are dealt with in their own category in the formulations herein. Nonionic surfactants are generally preferred in health care products since they are less harmful to humans compared to ionic surfactants. Thus, nonionic surfactants are preferred in the present invention. Commonly used nonionic surfactants are fatty acid ethoxylates esters and ethers, sorbitane monoesters, poloxamers and alkyl glucosides. The surfactant is preferably selected from fatty acid ethoxylates esters which are liquid or paste like at room temperature and HLB>10 but<20, to have good solubility in the system. More preferred surfactants are Polysorbate 20, Polysorbate 40, Polysorbate 60 and Polysorbate 80, with HLB about 15 to 17. The amount of surfactant soluble in the system will depend on the combination of the other components but preferably the amount is less than about 10% to have good stability and water dispersion capacity of the microemulsion.

Fatty acid alkyl esters belong to the group natural low molecular weight amphiphiles, polar lipids, that are widely used in food, cosmetic and pharmaceutical applications. They are esters formed between fatty acids or fatty acid derivatives and low molecular weight alcohols. Glycerol esters are formed between glycerol and a fatty acid or fatty acid derivatives. The glycerol esters can be monoglycerol esters or polyglycerol esters. The present formulations are directed to the use of polyglycerol fatty acid esters (PG-FA). Polyglycerol esters are mixed partial esters formed by reacting polymerized glycerols with fatty acids or fatty acid derivatives. The degree of polymerization varies, and is specified by a number (such as tri-) that is related to the average number of glycerol residues per polyglycerol molecule. By varying the proportions as well as the nature of the fats or fatty acids to be reacted with the glycerol or polyglycerol, a large and diverse class of products may be obtained. The composition of the glycerol esters will depend on the source and the supplier of the material, because all commercially available reagents are not identical and the exact purity may vary depending on the manufacture process. A conventional composition used by industry to call the material monoester is that it should contain at least 80% monoester. In the present invention PG-FA is used as polar lipid and emollient in a microemulsion formulation.

The PG-FA microemulsion formulation is preferably moisturizing to prevent dehydration of the nose also when the air is dry. It is preferably mucoadehsive to stay in the nose. It is preferably sprayable forming a yet when administered using a spraying device. It has preferably good spreadability on the mucosa to have good coverage of the nasal cavity. It is preferably stable on storage at low temperature to have a good usability.

Polyglycerol fatty acid esters (PG-FA) are commercially available with different chain length of the polyglycerol and derived from various fatty acids, resulting in lipids with different amphiphilicity and melting temperature. A product with good usability is achieved with ingredients that do not crystallise at low temperature. A liquid polar lipid below room temperature is achieved by increasing the polarity of the head group and/or using an unsaturated fatty acid.

According to the present invention PG-FA based on unsaturated fatty acids are preferred derived from natural vegetable sources. Examples of glycerol esters included in this invention are polyglycerol monooleate, polyglycerol monolinoleate, polyglycerol monolinenoleate. Minor amounts of glycerol mono-, di-, and triglycerides, free mono-, di-, and triglycerides, free glycerol and polyglycerols, free fatty acids, and sodium salts of fatty acids may be present in the polyglycerol monoesters. The required purity for making the compositions in this invention is 80% monoester

By mixing commercially available polyglycerol fatty acid esters and selective type of oil this will result in an isotropic liquid, reverse water in oil microemulsion at certain temperature conditions. The reverse microemulsion is formed at room temperature and below if the fatty acid is unsaturated. The reason why reverse micelles are formed even without the hydrophilic substance are due to strong amphilicity of the PG-FA. Impurities in the raw material like water and other hydrophilic substances such as free glycerol in the technical quality of the raw material may also contribute to the driving force to form a self-organized structure. The PG-FA and oil systems can only dissolve a minor fraction of hydrophilic solvent such as water in the core of the reverse micelles. The micellar shape is affected (from spherical to elongated) by the length of the head group of the PG-FA, the length of the alkyl chain of the PG-FA, the concentration of PG-FA, the type of oil, the temperature and the amount of water. Liquid crystalline phases are also observed in these systems.

The type of polar lipids that were found to be most interesting was polyglycerol monooleates (PGMO), preferably chosen from polyglycerol-2-oleate, polyglycerol-3-oleate, polyglycerol-4-oleate, polyglycerol-5-oleate, polyglycerol-6-oleate or mixtures thereof. These lipids have a relatively low melting temperature and are liquid at room temperature (unsaturated fatty acid chains as well as longer glycerol chains (longer than mono) decrease the melting temperature). Lipids from several suppliers were tested. The oleate content preferred was above 80%, which is a conventional composition used by industry to call the material a monoester. The head group length distribution was not known on these technical grade substances. The manufacturers denoted them differently depending on the main chain length. Therefore, the group of substances are referred to as polyglycerol monooleate, PGMO, in the remaining text.

The oils preferred according to the present invention are vegetable oils of high purity that are liquid at room temperature and below, resulting in liquid low irritation and low sensitization microemulsions at room temperature and below.

The water sorption capacity, the mucoadhesiveness, of the PGMO and vegetable oil mixture is increased by addition of polar solvent such as propylene glycol, which also has humectant properties. The addition of surfactant with high HLB (>10 but<20) increase the capacity to solubilise more polar solvent in the system.

We have now, surprisingly, found that incorporation of a humectant in said moisturising microemulsion may promote mucoadhesion due to water sorption from the underlying tissue, and at the same time increase water content in said tissue. Incorporation of humectants in an emollient formulation may balance the water content in the tissue and surprisingly reduce, or eliminate, the stinging sensation frequently observed with mucoadhesive formulations.

Carefully selected mixtures of PGMO, vegetable oil and polar solvents, and optional surfactant and humectants, are used in this invention to obtain a composition that has low viscosity, low and high temperature stability, good spreading on mucosa, good mucoadhesiveness and good moisturising effect.

The PG-FA microemulsion formulation is moisturizing to prevent dehydration of the nose also when the air is dry.

The PG-FA microemulsion formulation is mucoadhesive to stay in the nose for long duration.

The PG-FA microemulsion formulation is sprayable forming a jet when administrated from a traditional nasal spray device.

The PG-FA microemulsion formulation has good spreadability on the mucosa to have good coverage of the nasal cavity.

The PG-FA microemulsion formulation is surface active to entrap foreign particles such as pollen, dust mite and dust from polluted air.

The PG-FA microemulsion formulation is stable also at low temperature to have a good usability.

The microemulsion according to the present invention may comprise:
1-98 wt % polar lipid, selected from polyglycerol fatty acid esters, preferably 20-50 wt %, preferably 25-40 wt %;
1-98 wt % emollient (oil), not being a polyglycerol fatty acid ester, preferably 10-80 wt %, preferably 20-73 wt %, preferably 30-70 wt %, preferably 33-70 wt %;
0.1-35 wt % polar solvent including water or water solution, preferably 0.5-30 wt %, preferably 1-30 wt %, preferably 1-25 wt %, preferably 5-25 wt %, preferably 5-20 wt %;
0-10 wt % surfactant, not being a polyglycerol fatty acid ester;
0-20 wt % compound chosen from the group consisting of humectant, not being a polar solvent; antioxidant, preferably tocopherol; complexing agent; and preservative, preferably 0-10 wt %, preferably 0-5 wt %. For example a lower limit of the amount of humectant may be 0.001 wt %, such as 0.01 wt %, which may result in ranges such as 0.001-5 wt %, or 0.01-5 wt %.

A 100% PGMO product will have limited mucoadhesivity and high viscosity (non-sprayable). 100% oil will have limited mucoadhesivity. Preferably at least 20% PGMO is required to obtain a vehicle that is dispersing water within a few minutes. Preferably at most 50% PGMO is required to have a sprayable composition. A lower amount is required if a high viscous polar lipid or a high viscous polar solvent is used. Persons skilled in the art will be able to adjust the composition depending on the components included. The temperature stability between 2 and 40° C. is generally good for the whole range of compositions.

According to one embodiment of the present invention the microemulsion or composition does not contain any active pharmaceutical agent.

The present microemulsion or a composition comprising said microemulsion may be used as a medicament. The present microemulsion or a composition comprising said microemulsion may be used for the treatment and prevention of dry mucosa or prevention of airborne particles reaching (topical) mucosal membranes of a mammal. The present microemulsion or a composition comprising said microemulsion may be administered to mucous membrane, preferably nasal mucosa, vaginal mucosa, rectal mucosa or buccal mucosa, preferably nasal mucosa. The administration may be done by applying the microemulsion or a composition comprising said microemulsion to the mucosa e.g. by spraying, squirting, or sprinkling. When the microemulsion or composition is applied to vaginal or rectal mucosa it may alternatively be applied by instillation, e.g. using an instillator device, or applicator device. The administration may be made by an applicator device which administers the microemulsion or composition to the mucous membrane. Said applicator devise may comprise the microemulsion as a kit, or the microemulsion may be a applied to the applicator device just prior to administration to the mucosa. The administration may be made by a nasal spray, which sprays, squirts or sprinkles the microemulsion to the nasal mucosa. The administration may be made by a buccal spray, which sprays, squirts or sprinkles the microemulsion to the buccal mucosa. The administration may be made by a vaginal spray, which sprays, squirts or sprinkles the microemulsion to the vaginal mucosa. The administration may be made by a rectal spray, which sprays, squirts or sprinkles the microemulsion to the rectal mucosa. Depending on the construction of the nasal, buccal, rectal or vaginal spray apparatus the liquid formulations may be administered as spray, squirts or sprinkles. The wording nasal, buccal, rectal or vaginal spray is not to be considered limited to providing a spray plume of the microemulsion upon use. The wording nasal, buccal, rectal or vaginal spray is to be considered as a nasal, buccal, rectal or vaginal application by a nasal, buccal, rectal or vaginal application device. The nasal, buccal, rectal or vaginal application device applies the microemulsion by spraying, squirting or sprinkling the emulsion to the mucous membrane.

The present invention relates also to a liquid composition comprising said microemulsion. The liquid composition is preferably in a form suitable for nasal, buccal, rectal or vaginal administration. The liquid composition is preferably sprayable. Herein the wording sprayable is considered to include also that the material is able to be squirted or sprinkled.

### EXAMPLES

### Compositions

The criteria for a stable microemulsions at room temperature was that the composition should be isotropic and clear directly after mixing all ingredients and after equilibration at room temperature and below (2-8° C. stability for at least one day was a requirement). The compositions were observed over several months.

All excipients were charged and mixed in the storage containers by shaking the container by hand in most cases. Vortex mixing was occasionally used. In a few cases, the samples were mixed using magnetic stirring. The samples below were mixed and observed directly and after storage for at least one week at room temperature (about 22° C.) and at refrigerator temperature (2-8° C.) for at least one day. The manufacture procedure used can be transferred to large scale manufacture using the same principles by technical persons skilled in the art.

Simple phase diagrams were made where the different components were mixed and observed visually at room temperature. A microemulsion can be formed by mixing polar lipid (PGMO) and oil at room temperature. Polar solvent(s) is used to increase the mucoadhesivness and the surfactant is used to increase the stability of the microemulsion.

In the examples the polar lipid is chosen from polyglycerol-2-oleate, polyglycerol-3-oleate, polyglycerol-4-oleate or polyglycerol-6-oleate. Most examples were made with polyglycerol-2-oleate, polyglycerol-3-oleate, and polyglycerol-4-oleate.

In the examples the oil is chosen from sesame oil, maize oil, sunflower oil, olive oil, MCT, paraffin oil, and Canola oil. Most examples were made with sesame oil, sunflower oil or olive oil as emollient.

In the examples the polar solvent is selected from glycerol, propylene glycol, caprylyl glycol, propanediol, pentylene glycol, and polyethylene glycol, whereof all have humectants properties. The polar solvent can also be water or water solutions. Most examples were made with glycerol and propylene glycol as polar solvent.

In the examples the surfactant is Polysorbate 80 or Polysorbate 60. Most examples were made with Polysorbate 80 as surfactant.

### Example 1 Compositions Temperature Stability

Some compositions from EP1648412 were mixed and investigated at temperatures equal or below 40° C. for at least one day. The temperature where the samples phase separated or if crystals were visible was recorded. Similar experiments were made with mixtures of PGMO, oil, polar solvent(s), and surfactant. By carefully selecting the components, microemulsions in the PGMO system are stable at about 2-8° C. and above and the GMO/sesame oil/Polysorbate 80/PG/PEG400/saline system is stable at about 15° C. and above. Low amount of PGMO (<20%) is difficult to combine with high amount of polar solvent (>5%) without optimal concentration of surfactant (about 5%).

### Compositions (w/w %)

| | | | | | PEG | | | | Appearance | | |
|---|---|---|---|---|---|---|---|---|---|---|---|
| | GMO | PGMO# | PG | Glycerol | 400 | Oil* | Water | PS80 | 2-8° C. | ~23° C. | 40° C. |
| Sample 1 | 36 | — | 24 | — | 19 | 12 | 5 | 4 | Crystals | Clear | Clear |
| Sample 2 | 38 | — | 26 | — | 20 | 12 | — | 4 | Crystals | Clear | Clear |
| Sample 3 | 37 | — | 26 | — | 20 | 12 | 1 | 4 | Crystals | Clear | Clear |
| Sample 4 | 36 | — | 24 | 5 | 19 | 12 | — | 4 | Solid | Clear | Clear |
| Sample 5 | — | 42 | 11 | — | — | 42 | — | 5 | Clear | Clear | Clear |
| Sample 6 | — | 42 | 11 | — | — | 40 | 2 | 5 | Clear | Clear | Clear |
| Sample 7 | — | 34 | 7 | — | — | 51 | 3 | 5 | Clear | Clear | Clear |
| Sample 8 | — | 50 | — | — | — | 50 | — | — | Clear | Clear | n.i. |
| Sample 9 | — | 37 | 10 | — | — | 53 | — | — | Clear | Clear | Clear |
| Sample 10 | — | 35 | 5 | 15 | — | 40 | — | 5 | Clear | Clear | Clear |
| Sample 11 | — | 30 | 2 | 10 | — | 55 | — | 3 | Clear | Clear | Clear |
| Sample 12 | — | 35 | 5 | — | — | 60 | — | — | Clear | Clear | Clear |
| Sample 13 | — | 30 | 5 | 16 | — | 43 | 2 | 4 | Clear | Clear | Clear |
| Sample 14 | — | 25 | 5 | — | — | 65 | — | 5 | Clear | Clear | Clear |
| Sample 15 | — | 45 | 5 | — | — | 35 | — | 5 | Clear | Clear | Clear |
| Sample 16 | — | 33 | — | — | — | 67 | — | — | Clear | Clear | n.i. |
| Sample 17 | — | 35 | 7.5 | — | — | 52.5 | — | 5 | Clear | Clear | Clear |
| Sample 18 | — | 18 | 5 | 4 | — | 73 | — | — | Clear | Clear | n.i. |
| Sample 19 | | 20 | — | — | — | 80 | — | — | Clear | Clear | n.i. |
| Sample 20 | — | 35 | 7.5 | — | — | 57.5 | — | — | Clear | Clear | Clear |
| Sample 21 | — | 35 | 7.5 | — | — | 52.5 | — | 5- | Clear | Clear | Clear |
| Sample 22 | — | 35 | 7.5 | — | — | 47.5 | — | 10 | Clear | Clear | Clear |
| Sample 23 | — | 35 | — | — | — | 60 | — | 5 | Clear | Clear | Clear |
| Sample 24 | — | 35 | 12.5 | — | — | 47.5 | — | 5 | Clear | Clear | Clear |

GMO = glycerol monooleate,
PG = propylene glycol,
PEG 400 = polyethylene glycol 400,
PS80 = Polysorbate 80,
n.i. = not investigated
#Variation between polyglycerol-2 oleate, polyglycerol-3-oleate and polyglycerol-4-oleate
*Variation and combination between olive oil, sesame oil and medium chain triglycerides

### Example 2 Low Temperature Stability

Five test formulations, comprising two alternative monoglycerides (GMO and PGMO) and variations in polar solvents were manufactured. The details of these samples are given in Example 1. DSC (Differential scanning calorimeter, Mettler Toledo) was used to determine thermotropic phase transitions in the range from −50 to +40° C. The DSC was calibrated for heat flow and temperature using indium (m.p. 156.6° C.; H=28.45 J/g) each time before starting the measurement. The reference pan was an empty sealed pan during all experiments. Samples were placed into 40 μl aluminum pans and sealed hermetically by using a crucible sealing press. Dry nitrogen gas flowing at 80 ml/min was used to purge the furnace chamber. Three scan rates were selected for the samples: 5° C./min, 1° C./min and 0.2° C./min. Two subsequent sets of experiments were performed.

In the first set of experiments the samples were heated to +40° C., then cooled down to −40° C. and again heated to +40° C. for scan rate of 5° C./min and 1° C./min for all the samples. For scan rate of 0.2° C./min the samples were cooled down to −40° C. and then heated to +40° C. In these DSC results some peaks were overlapping and to have a clear idea about the peaks the temperature ranges were changed. In the second set of experiments the samples were heated to +40° C., and then cooled down to −50° C. and held for 10 minutes at this temperature and then again heated to +40° C. with a scan rate of 5° C./min and 1° C./min. For scan rate of 0.2° C./min the samples were cooled down to −50° C. and held for 10 minutes and then heated to +40° C. The same samples were used for different scan rates and various temperature ranges.

The melting temperature is significantly lower for the PGMO microemulsion system compared to the GMO microemulsion system. The water content has only a minor effect on the same melting temperature.

### DSC Scans

| | Formulations | | | | | | |
|---|---|---|---|---|---|---|---|
| | Sample 1 | Sample 1 | Sample 2 | Sample 3 | Sample 5 | Sample 6 | Sample 17 |
| Scanning | Endset | Endset | Endset | Endset | Endset | Endset | Endset |
| rate | Temp | Temp | Temp | Temp | Temp | Temp | Temp |
| (° C./min) | (° C.) | (° C.) | (° C.) | (° C.) | (° C.) | (° C.) | (° C.) |
| 0.2 | 15.4 | — | 16.9 | 16.8 | 8.5 | 8.0 | — |
| 1 | 14.2 | 10.9 | 15.5 | 15.6 | 7.1 | 6.4 | −13.1 |
| 5 | 14.9 | 11.8 | 17.1 | 17.1 | 0.4 | 0.6 | −13.0 |

### Example 3 Viscosity

The formulations should preferably be low viscous and sprayable to be easy to administer to the nose and to fast spread in the nostrils. The viscosity of the microemulsions are dependent on the composition. Four different methods were used to determine the sprayability of the composition. (1). Flow, one drop of product was placed on a glass plate which was tilted 90° for 10 seconds. The length of the drop was measured. (2) Viscosity measured with a Rheometer in a shear sweep, viscosity at shear rate 117 s⁻¹ was compared (all formulations tested were Newtonian liquids). (3) Product filled in bottle with a traditional spray pump for liquids. The force that needed to press the sample out of the spray outlet was estimated (easy or hard). (4). Visual viscosity by tilting the beaker with the sample compared to the other samples investigated (low, medium, high). The combination of raw materials need to be carefully selected to be able to get a moisturizing, mucoadhesive, sprayable, homogeneous liquid at temperature between 5 and 40° C.

### Compositions (w/w %) and Viscosity

| | | | | | | | | | Viscosity | | | |
|---|---|---|---|---|---|---|---|---|---|---|---|---|
| | | | | | | | | | | App | | |
| | GMO | PGMO# | PG | Glycerol | PEG400 | Oil* | Water | PS80 | Flow | visc. | | |
| | wt % | wt % | wt % | wt % | wt % | wt % | wt % | wt % | (cm) | (Pas) | Pump | Visual |
| Sample 1 | 36 | — | 24 | — | 19 | 12 | 5 | 4 | 4.3 | 0.11 | Easy | Low |
| Sample 2 | | 45 | 6 | | | 45 | | 4 | 2.7 | 0.26 | Hard | Medium |
| Sample 3 | | 35 | 5 | 10 | | 45 | | 5 | 2.7 | 0.37 | Hard | High |
| Sample 4 | | 45 | 6 | | | 45 | | 4 | 2.9 | 0.31 | Hard | Medium |
| Sample 5 | | 35 | 5 | 10 | | 45 | | 5 | 3.0 | 0.39 | Hard | High |
| Sample 6 | | 71 | 5 | | | 18 | 1 | 5 | 2.6 | 0.38 | Hard | High |
| Sample 7 | | 50 | 10 | | | 35 | | 5 | 3.3 | 0.15 | Easy | Low |
| Sample 8 | | 35 | 7.5 | | | 52.5 | | 5 | 3.4 | 0.18 | Easy | Low |
| Sample 9 | — | 35 | 5 | | | 55 | | 5 | 4.1 | 0.11 | Easy | Low |
| Sample 10 | — | 35 | 7.5 | — | — | 52.5 | — | 5 | — | 0.24 | Hard | Medium |

#Variation between polyglycerol-2 oleate, polyglycerol-3-oleate and polyglycerol-4-oleate
*Variation and combination between olive oil, sesame oil and medium chain triglycerides

### Example 4 Mucoadhesive

The mucoadhesiveness of products were studied in visual colouration experiments. A water solution with blue dye was mixed. Thin product films were made by dropping product on a glass plate (about 0.5 ml product). All products studied are spreading quickly on hydrophilic glass surfaces and form a thin film. The uptake/spreading of the water based dye in the thin product films were observed immediately after addition of a drop of dye (about 20 μl) and followed over the next hour. The experiment gave information on how fast water is dispersed in the system. A composition that can disperse water fast is more mucoadhesive since the driving force to absorb water is strong. A non-water sorbing product will not be mucoadhesive.

One composition with GMO (from EP1648412) was mixed and studied, sample 1. 20 to 50% PGMO and various types of oils (e.g. sesame oil, olive oil, MCT, paraffin, Canola oil) were mixed and studied (sample 2-16). Selected compositions with PGMO, oil, surfactant and polar solvent(s) were mixed (sample 17-26). As references, the spreading of the water based dye to sesame oil (Ref 1) and polar solvents (Ref 2-5) were studied.

This experiment shows that the water sorption capacity of the microemulsion can be varied from very fast to slow depending on the composition. The blue dye is spreading very fast into sample 1, within seconds. The spreading into the sample 2-13 is slower, within 15 minutes. It does not spread significantly in the microemulsion made with paraffin oil, sample 14-16. The spreading of the water in the PGMO mixtures with polar solvent(s) and surfactant, sample 17-25, are slower than sample 1, and faster than sample 2-16, and will depend on the composition. Formulations with polar solvent in combination with surfactant gives the fastest and most even sorption, i.e., the highest mucoadhesion. The water based dye do not spread into the sesame oil film (Ref 1). The water based dye is spreading within minutes in water and polar solvents such as propylene glycol, pentylene glycol, water or glycerol (Ref 2-5).

### Compositions (w/w %)

| | | | | | | Paraffin | Polar | |
|---|---|---|---|---|---|---|---|---|
| | GMO | PGMO | PG | PEG400 | Oil | oil | solvent* | PS80 |
| Sample 1 | 36 | — | 24 | 19 | 12 | 5 | 4 | 36 |
| Sample 2- | — | 20, 35 | — | — | 80, 65 | — | — | — |
| 13 | | or 50 | | | or 50 | | | |
| Sample | — | 20, 35 | — | — | — | 80, 65 | — | — |
| 14-16 | | or 50 | | | | or 50 | | |
| Sample | — | 20-50 | — | — | 20-80 | — | 0-20 | 0-10 |
| 17-30 | | | | | | | | |
| Ref 1 | — | — | — | — | 100 | — | — | — |
| Ref 2 | — | — | 100 | — | — | — | — | — |
| Ref 3-5 | — | — | — | — | — | — | 100 | — |

PG = propylene glycol,
PEG 400 = Polyethylene glycol 400,
PS80 = Polysorbate 80,
Oil = sesame oil, olive oil, MCT or Canola oil
Polar solvent* = polar solvent such as water, glycerol, propylene glycol, pentylene glycol, caprylyl glycol, and polyethylene glycol 400.

### Example 5 Water Sorption Capacity

An oil solution with red dye and a water solution with blue dye were made. 20 μl of the dyed solutions were added to about 2 ml product (corresponding to approximately 1% oil and water, respectively).

Samples were mixed according to the table below (w/w %).

| | | | Polar solvent | |
|---|---|---|---|---|
| | | | including | |
| | | | water/water | |
| | PGMO | Oil | solutions | Surfactant |
| Samples | 1-70 | 15-99 | 0-25 | 0-10 |

Sesame oil was used as reference. Both the red dyed oil and the blue dyed water spread in the microemulsion formulations spontaneously. PGMO and oil mixtures can dissolve very low amount of water at low concentration PGMO and more at higher concentration PGMO 20% PGMO). Sesame oil is only oil continuous and cannot absorb water. Only the red dye is spreading spontaneously in the pure oil.

The amount of water that is soluble in the microemulsion is related to the micellar size. In the simplest system, PGMO and oil, the micellar size does not increase with increasing PGMO concentration but the number of micelles increase (discussed by Shrestha et al, 2010). Larger amount of water can be dissolved in more micelles but the total amount is low (maximum about 1 wt %).

If surfactant is added to the system, more polar solvent can be dissolved in the oil and PGMO microemulsion. The amount of water that can be dissolved in the micelles is related to the micellar shape which is related to the molecular packaging of the components (PGMO, oil, polar solvent, surfactant). The molecular packing of the components depends on the quality of the components, the ratio between the components and the temperature and has to be carefully selected. ¹H-NMR (Nuclear Magnetic Resonance) of Sample 1 and 7 in Example 1 and all the ingredients were collected and the diffusion constants were calculated. The self-diffusion coefficients shows that the water is in the core of the micelles. There was no difference between the two samples and hence the shape and size of the micelles are in the same size range showing the excellence of the system selected for the purpose of the invention

### Example 6 Water Sorption Capacity

The tendency to take up water by the products was also studied gravimetrically with time after storing the products in well-defined relative humidity chambers. The study was made at 43% RH (saturated solution with potassium carbonate put in a desiccator at about 23° C.) and 97% RH (saturated solution with potassium sulphate put in a desiccator at about 23° C.). Note that 97% RH was selected to avoid condensation. Long experimental time was required to be able to make observations under equilibrated conditions.

### Compositions (w/w %) and Water Uptake

| | | | | | | | | | Relative | | Relative | |
|---|---|---|---|---|---|---|---|---|---|---|---|---|
| | | | | | | | | | increase | | increase | |
| | | | | | | | | | (%) | | (%) | |
| | | PGMO | | | PEG | | | | 43% RH | | 97% RH | |
| | GMO | (4GO) | PG | Glycerol | 400 | Oil* | Water | PS80 | after | | after | |
| | wt % | wt % | wt % | wt % | wt % | wt % | wt % | wt % | 18 d | 53 d | 18 d | 53 d |
| Sample 1 | 36 | — | 24 | — | 19 | 12 | 5 | 4 | 1.0 | 0 | 13.7 | 25.4 |
| Sample 2 | — | 35 | 7.5 | — | — | 52.5 | — | 5 | 1.0 | 0.3 | 7.6 | 11.1 |

*Variation and combination between olive oil, sesame oil and medium chain triglycerides

### Compositions (w/w %) and Water Uptake

| | | | | | | Relative increase |
|---|---|---|---|---|---|---|
| | | | | | | (%) 97% RH |
| | PGMO# | PG | Glycerol | Oil* | PS80 | after 7 days |
| | wt % | wt % | wt % | wt % | wt % | % |
| Sample 1 | 20 | — | — | 80 | — | 0.5 |
| Sample 2 | 35 | 7.5 | — | 57.5 | — | 1.2 |
| Sample 3 | 35 | — | — | 60 | 5 | 1.0 |
| Sample 4 | 35 | 7.5 | — | 52.5 | 5 | 1.4 |
| Sample 5 | 35 | 12.5 | — | 47.5 | 5 | 2.0 |
| Sample 6 | 35 | 5 | 10 | 45 | 5 | 1.1 |
| Sample 7 | 35 | 5 | 15 | 40 | 5 | 1.2 |
| Sample 8 | 35 | 7.5 | — | 47.5 | 10 | 1.7 |

Formulations absorb large amounts of water at 97% RH and low amount at 43% RH. The microemulsions phase separate into two phases at 97% RH. This was apparent already after a few days, however this did not stop the formulations from keep absorbing water. This is an indirect measure of the strong hygroscopic behavior of the products. Polar solvent in combination with surfactant increase the sorption capacity of the formulation, i.e., the mucoadhesion.

### Example 7 Mucoadhesion

Mucoadhesion is related to the ability to adhere to the mucosa. This can be achieved through water sorption from tissue to the applied application. Hygroscopic formulations absorbs water molecules but at different timescales depending on the composition. Tensile strength using micro balance texture analyzer was used to measure the mucoadhesion. The work needed to separate two layers from each other is calculated as well as the peak measured force.

The microemulsions show quite strong mucoadhesion, with similar tensile strength values. To get some reference values, the mucoadhesion of olive oil, propylene glycol and PGMO were also tested as well as the adhesion of microemulsions towards water. Propylene glycol was chosen to determine if humectant properties on its own had any influence on the adhesion strength.

The comparison results on olive oil and propylene glycol showed significantly lower values than the two formulations Sample 1 and Sample 17. PGMO is intrinsically very sticky and attaches strongly to most surfaces. The adhesion of the formulations towards water was significantly lower compared to mucin.

| Upper | | Mucoadhesion | Peak force |
|---|---|---|---|
| sample | Lower sample | (kg s) | (kg) |
| Mucin | Sample 1 | 0.18 ± 0.18 | 0.89 ± 0.11 |
| Mucin | Sample 17 | 0.21 ± 0.16 | 0.88 ± 0.21 |
| Water | Sample 1 | 0.067 ± 0.033 | 0.65 ± 0.24 |
| Water | Sample 17 | 0.090 ± 0.034 | 0.76 ± 0.20 |
| Water | Water | 0.017 ± 0 | 0.12 ± 0.01 |
| Mucin | Olive oil | 0.089 ± 0.016 | 0.79 ± 0.14 |
| Mucin | Propylene Glycol | 0.051 ± 0.009 | 0.50 ± 0.05 |
| Mucin | PGMO | 0.75 ± 0.31 | 2.4 ± 0.26 |
| Dry paper | Dry paper | 0 | 0.006 |

### Example 8 Emollient Effect

Emollients are materials that smooth the surface and make the surface look uniform to the eye and silkier to touch. Emollients are lipophilic and may produce an occlusive film which may promote a steeper a_{w} gradient in tissue i.e. the emollient becomes a barrier to the outside which hampers evaporation from the superficial layer in tissue. The occlusive effect of microemulsions was determined and compared to the occlusive effect of sesame oil.

### Compositions (w/w %)

| | | | | | | | Saline | |
|---|---|---|---|---|---|---|---|---|
| | GMO | PGMO | PG | Glycerol | PEG400 | Oil | water | PS80 |
| Sample 1 | 36 | — | 24 | — | 19 | 12 | 5 | 4 |
| Sample 2 | — | 35¹ | 5 | — | — | 55 | — | 5 |
| Sample 3 | | 35² | 12.5 | — | — | 47.5 | — | 5 |
| Sample 4 | — | 35³ | 5 | 15 | — | 40 | — | 5 |
| Reference 1 | — | — | — | — | — | 100 | — | — |
| Reference 2 | — | — | — | — | — | — | — | — |

PG = propylene glycol,
PEG 400 = Polyethylene glycol 400,
PS80 = Polysorbate 80,
Oil = sesame oil or olive oil.
PGMO = ¹polyglycerol-3-oleate, ²polyglycerol-4-oleate, ³polyglycerol-2-oleate

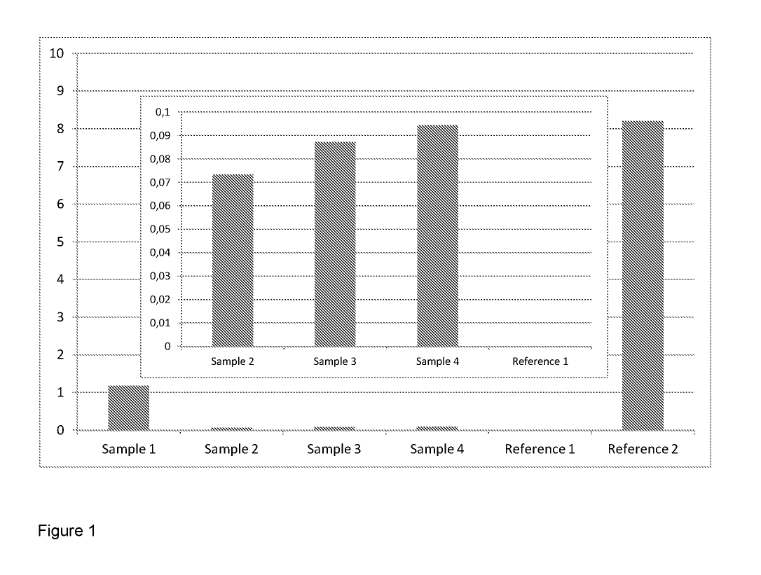

The microemulsion is forming a thin film on a hydrophilic surface, e.g. the nasal mucosa or a gelatine hydrogel. In this experiment a gelatine hydrogel was used as hydrophilic water containing surface. The water evaporation can be measured with time gravimetrically and the occlusive effect of the product determined. A thin film of product was applied on the hydrogel and the water evaporation was measured over time. The water soluble component (propylene glycol, glycerol and water solution) will dissolve in the gel with time or evaporate (propylene glycol and water solution) and the product film on top of the hydrogel will be enriched with the emollients. The results are shown in FIG. 1 illustrated as gravimetric evaporation (change in mass as slope per day) from a thin film of product on a gelatin hydrogel at steady state (slope). Surrounding relative humidity about 20 to 40% RH. Reference 1 is a sesame oil product that is occlusive. Reference 2 is not covered with a product and is non-occlusive. Sample 1 contains high amount of evaporating and non-evaporating polar solvents, 48 wt %, and show some occlusivity. Sample 2 contains low amount of polar solvent, 5 wt %, and show high occlusivity. Sample 3 and 4 which contain 12.5 wt % and 20 wt % polar solvents, respectively, are less occlusive than Sample 2 but more than Sample 1.

### Example 9 Spreading

To be able to apply a nasal product, the viscosity should preferably be low to be dropped or sprayed into the nostrils. However, in order to keep the product retained in the nasal cavity for absorption to occur, the product should preferably be more viscous. Water based products are easily cleared by the hydrophilic mucocilary clearance system and require mucoadhesive properties to have a technical benefit. The products in this invention are sprayable forming a fine yet. They are, low viscous oily liquids that spread easily to cover the whole nasal cavity for optimal protection. The challenge with the microemulsions is that they should not be spreading so quickly that they reach the throat to be swallowed and tasted or to leak though the nose tip after administration.

The wetting behaviour of a product will show how efficient the product is when it comes to its spreading on the mucosa. This was determined using contact angle measurements and spreadability tests. Glass slides treated with ethanol were used as hydrophilic surfaces. Glass slides coated with a thin paraffin layer were used as hydrophobic surface. The contact angle was measured after dropping 20 μl product on a glass plate and measuring the contact angle after 15 minutes using a Drop Shape Analyzer. The average value of six measurements per product was calculated. The spreadability was measured by determining the diameter of the product drop after 15 minutes using a ruler. The average value of six measurements per product was calculated.

All microemulsions tested have better wetting behaviour compared to saline solutions and vegetable oil. There is no significant difference between the wetting behaviour of microemulsions made by GMO (from EP1648412) and microemulsions formed with PGMO and vegetable oil.

### Compositions Studied (w/w %):

| | | | | | Sesame | Saline | |
|---|---|---|---|---|---|---|---|
| | GMO | PGMO | PG | PEG400 | oil | water | PS80 |
| Sample 1 | 36 | — | 24 | 19 | 12 | 5 | 4 |
| Sample 2 | 38 | — | 26 | 20 | 12 | — | 4 |
| Sample 3 | — | 42 | 11 | — | 42 | — | 5 |
| Reference 1 | — | — | — | — | 100 | — | — |
| Reference 2 | — | — | — | — | — | 100 | — |

PGMO = Polyglycerol-3-oleate,
PG = propylene glycol,
PEG 400 = Macrogol 400,
PS80 = Polysorbate 80

Contact angle:

| | Hydrophilic | Hydrophobic |
|---|---|---|
| | glass | glass |
| Sample 1 | 6 ± 1 | 43 ± 3 |
| Sample 2 | 6 ± 2 | 31 ± 1 |
| Sample 3 | 7 ± 1 | 30 ± 8 |
| Reference 1 | 16 ± 2 | 39 ± 2 |
| Reference 2 | 12 ± 1 | 63 ± 3 |

### Example 10 Spreadable

The microemulsions are liquids that spread easily on both polar and apolar surfaces since they are amphiphilic preparations. Spreading on surfaces (wetting) is dependent on the surface activity of the formulation and the viscosity. The surface activity was determined by measuring the surface tension (at the formulation-air interface) using a drop volume tensiometer. A drop is dispersed through a capillary and held at the tip. The drop is then optically observed and the surface tension is calculated from the shape of the drop.

Compared with the volume phase, a molecule at the surface of a liquid meets fewer molecules with which it can form interactions. Presence at the surface is therefore less beneficial from an energy point of view. A liquid therefore assumes the smallest possible surface area without the action of external force. Work must be done in order to increase the area of the surface. The work which is required to increase the size of the surface of a phase is referred to as the surface tension (measure of work per unit area or force per wetted length, unit mN/m). Surfactants are so-called surface-active substances, as they reduce the surface tension of liquids or the interfacial tension of two-phase systems due to adsorption at the surface or interface, respectively.

Sample 1 from Table 1 had a surface tension of 29.4±0.2 mN/m and Sample 17 had a surface tension of 29.7±0.1 mN/m. Pure water has a surface tension of 72.0 mN/m at 25° C. Propylene glycol has a surface tension of 45.6 mN/m and glycerol 76 mN/m at 25° C. The microemulsions are surface active.

### Example 11 Entrapment

Surface active preparations can entrap pollen and microorganisms due to their amphiphilic properties, and thereby prevent the particles from reaching the mucosa and inhibit the release of allergens or start an infection. Water was charged in a Petri dish. Microemulsion (Sample 1 and 5 in Example 1) was charged in other Petri dishes. Pollen particles were placed onto the surface. The pollen particles cannot spread on the surface with microemulsion. They are entrapped. The pollen particles spread to a thin film on the water surface.

### Example 12 Clinical Study. Moisturizing and Mucoadhesive but Less Stinging

If formulation water sorption and resulting dehydration of the mucosa is very fast this may lead to an unpleasant stinging sensation upon application. Balanced emollient effect of the formulation by the occlusive effect of the lipophilic substances and incorporation of a humectant in the microemulsion may still promote mucoadhesion due to water sorption from the underlying tissue, and at the same time increase water content in said tissue. This may reduce, or eliminate, the stinging sensation frequently observed with mucoadhesive formulations.

The aim of this study was to evaluate the usability of microemulsions. The compositions were as follows and the products were filled into brown glass vial with a pump.

### Compositions:

| | | | | | | | | Menthol, |
|---|---|---|---|---|---|---|---|---|
| | GMO | PGMO | PG | PEG400 | Oil | Saline | PS80 | eucalyptus |
| | % (w/w) | % (w/w) | % (w/w) | % (w/w) | % (w/w) | % (w/w) | % (w/w) | % (w/w) |
| Sample 1 | 36 | — | 24 | 19 | 12 | 5 | 4 | <0.01 |
| Sample 2 | — | 35 | 5 | — | 55 | — | 5 | — |

PG = propylene glycol,
PEG 400 = Polyethylene glycol Macrogol 400,
PS80 = Polysorbate 80
Oil = sesame oil or olive oil.
PGMO = polyglycerol-3-oleate

The study was performed as a user study where healthy volunteers were using the products for about three days, one product at the time. The instructions to the users were to test one product at a time for at least 3 days and then give an opinion about their properties in a questionnaire.

Ten detailed questions about the product were first asked and finally the total experience was given.

The study was completed by 17 volunteers, both male and female, age between 13 and 63 years. The volunteers used the product according to the instruction, for at least three days per product and at least one time a day. The results were not evaluated on how many times per day the products were used by the volunteers.

The product is designed to be mucoadhesive, and a prerequisite for adhesion is that there is a water gradient between the product and the mucosa. The stinging in the nose is correlated to this water transport from the mucosa to the product film. If there is a nasal inflammation ongoing, this could be advantageous effect but if not it may only be irritating. The work to reformulate the product has focused on decreasing this water pulling effect but still remaining the mucoadhesivness of the product.

There was no difference in how the moisturising effect was experienced and the duration seems to be equally long. However sample 2 was significantly less irritating compared to sample 1 and significantly better appreciated in total.

1. A microemulsion comprising
1-98 wt % of at least one polar lipid selected from polyglycerol fatty acid esters;
1-98 wt % of at least one emollient, not being a polyglycerol fatty acid ester;
0.1-35 wt % of at least one polar solvent; and
0-10 wt % of a surfactant, not being a polyglycerol fatty acid ester.

2. The microemulsion according to claim 1, wherein the at least one polar lipid is selected from fatty acid esters of diglycerol to hexaglycerol, preferably fatty acid esters of diglycerol to pentaglycerol, preferably fatty acid esters of diglycerol to tetraglycerol.

3. The microemulsion according to claim 1, wherein the fatty acid esters of the polyglycerol fatty acid esters are chosen from oleate, linoleate, and linenololeat, preferably chosen from monooleate, monolinoleate, and monolinenololeat, preferably monooleate.

4. The microemulsion according to claim 1, wherein the polyglycerol fatty acid esters are present in an amount of 20-50 wt %, preferably 25-40 wt %.

5. The microemulsion according to claim 1, wherein the at least one polar solvent is chosen from hydrophilic solvents, preferably water or water solution; glycerol; low molecular weight glycerol derivatives; propylene glycol; low molecular weight derivatives of propylene glycol; low molecular weight alcohol, preferably ethanol, isopropyl alcohol and transcutol; butylene glycol; pentylene glycol; hexylene glycol; dipropylene glycol; propanediol; panthenol; propylene carbonate; and polyethylene glycol.

6. The microemulsion according to claim 1, wherein the at least one polar solvent is present in an amount of 0.5-30 wt %, preferably 1-30 wt %, preferably 1-25 wt %, preferably 5-25 wt %, preferably 5-20 wt %.

7. The microemulsion according to claim 1, wherein the at least one emollient is selected from fat, animal or vegetable oils, vegetable butters, wax, silicones, petrolatum and mineral oil; preferably mineral oil, silicone oils, animal or vegetable oils; preferably vegetable oils.

8. The microemulsion according to claim 7, wherein the at least one emollient is selected from the group consisting of low molecular weight linear polysiloxanes and cyclic dimethyl polysiloxanes; liquid paraffin; lanolin oil; vegetable triglyceride containing oil selected from almond, avocado, canola, castor, coconut, corn, evening primrose, jojoba, linseed, olive, safflower, sunflower, sesame, soybean, and wheat germ oil; and extractions of triglycerides with 8 to 10 carbons in the chain.

9. The microemulsion according to claim 1, wherein the at least one emollient is present in an amount of 10-80 wt %, preferably 20-73 wt %, preferably 30-70 wt %, preferably 33-70 wt %.

10. The microemulsion according to claim 1, wherein the at least one surfactant is selected from nonionic surfactants; preferably hydrophilic surfactants; preferably fatty acid ethoxylates esters and ethers, sorbitane monoesters, poloxamers and alkyl glucosides; preferably polysorbate 20, 40, 60 or 80; preferably polysorbate 60 or 80; preferably polysorbate 80.

11. The microemulsion according to claim 1, wherein the at least one surfactant is present in an amount of 0-7 wt %.

12. The microemulsion according to claim 1, which may further comprise compound chosen from a humectant, not being a polar solvent; an antioxidant, preferably tocopherol; a complexing agent; and a preservative.

13. The microemulsion according to claim 12 wherein the humectant is selected from the group urea, aloe vera, pidolic acid, alfa-hydroxy acids, sorbitol, xylitol, manitol and natural polymers having humectant properties, preferably the natural polymer is hyaluronic acid.

14. The microemulsion according to claim 12, wherein the at least one humectant is present in an amount of 0-20 wt %, preferably 0-10 wt %, preferably 0-5 wt %.

15. A microemulsion according to claim 1, being an oil-continuous isotropic solution with no aggregates; or having discrete reverse micelles with a hydrophilic core and a hydrophobic continuous phase.

16. A microemulsion according to claim 1 for use as a medicament.

17. A microemulsion according to claim 1 for use in the treatment and prevention of dry mucosa or prevention of airborne particles reaching topical mucosal membranes of a mammal.

18. A microemulsion for use according to claim 17, wherein the microemulsion is administered in the form of a nasal, buccal, rectal or vaginal application; preferably nasal spray, buccal spray, rectal spray or vaginal spray.

19. A liquid composition comprising the microemulsion according to claim 1.

20. The liquid composition according to claim 19, wherein said composition is in a form suitable for nasal, buccal, rectal or vaginal administration.

21. The liquid composition according to claim 19, wherein said composition is sprayable.

22. An applicator device comprising the microemulsion according to claim 1.

23. The applicator device according to claim 22, being a nasal applicator device, a buccal applicator device, an rectal applicator device or a vaginal applicator device; preferably being a nasal spray device, a buccal spray device, a rectal spray device or a vaginal spray device.
